(19) United States
(12) Patent Application Publication
(10) Pub. No.: US 2023/0190836 A1
(43) Pub. Date: Jun. 22, 2023

(54) PHOTOSYNTHETICALLY CONTROLLED SPIRULINA EXTRACTS FOR TREATING THE CYTOKINE STORM SYNDROME

(71) Applicant: Vaxa Technologies Ltd, Rosh Pinna (IL)

(72) Inventors: Isaac BERZIN, Jerusalem (IL); Ohad BASHAN, Sde Varburg (IL)

(73) Assignee: Vaxa Technologies Ltd, Rosh Pinna (IL)

(21) Appl. No.: 17/925,986

(22) PCT Filed: May 19, 2021
(86) PCT No.: PCT/IL2021/050577

§ 371 Date: Nov. 17, 2022

Related U.S. Application Data
(60) Provisional application No. 63/142,502, filed on Jan. 28, 2021.
(60) Provisional application No. 63/026,764, filed on May 19, 2020.

(51) Int. Cl. A61K 35/748; A61K 31/4025; A61K 31/047; A61K 31/7076; C12N 1/20

(52) U.S. Cl. CPC A61K 35/748; A61K 31/4025; A61K 31/047; A61K 31/7076; C12N 1/20

(57) ABSTRACT

Spirulina extracts and/or fractionated compounds thereof, sublingual spray formulation thereof, spraying devices for using the formulation, as well as methods of preparing and using the spirulina extracts to treat TNF-α related inflammation are provided. The spirulina extracts are prepared by water extraction of Arthrospira spp. biomass that is cultivated under controlled, ultra-high-density conditions with strong UV illumination and strong continuous mixing, and are characterized by high levels of c-phycocyanin, sorbitol and adenosine derivates—which were found to have a strong low-dose effect of reducing TNF-α secretion. The spirulina extracts may correspondingly be used to prevent or alleviate cytokine storm related to various infections or autoimmune diseases.

### BACKGROUND OF THE INVENTION

### 1. Technical Field

The present invention relates to the field of photosynthetically controlled spirulina extracts, and more particularly, to treating the cytokine storm syndrome using the spirulina extracts.

### 2. Discussion of Related Art

Inflammatory diseases are common due to external infections or auto-immune diseases. Specifically, cytokine storm (CS), or hypercytokinemia, triggered by the macrophage activation syndrome (MAS) involves high macrophage- and monocyte-induced tumor necrosis factor (TNF)-α levels and can lead to life-threatening conditions.

### SUMMARY OF THE INVENTION

The following is a simplified summary providing an initial understanding of the invention. The summary does not necessarily identify key elements nor limit the scope of the invention, but merely serves as an introduction to the following description.

One aspect of the present invention provides a spirulina extract comprising a water-based extract of Arthrospira spp., cultivated under photosynthetically controlled conditions to yield upregulated bio-active compounds comprising c-phycocyanin, sorbitol and adenosine derivates, wherein the spirulina extract has a concentration smaller than 10 μg/ml and is effective as an anti-inflammatory agent.

One aspect of the present invention provides a sublingual spray formulation comprising the spirulina extract and/or fractionated compounds thereof, at a concentration smaller than 10 μg/ml and spraying devices configured to administer the sublingual spray formulation to the sublingual mucosa.

One aspect of the present invention provides a method of preparing a spirulina extract, the method comprising: cultivating Arthrospira spp. cyanobacteria, cultivated under photosynthetically controlled conditions to yield upregulated bio-active compounds comprising c-phycocyanin, sorbitol and adenosine derivates, preparing a water-based extract of the cultivated Arthrospira spp. cyanobacteria to yield a spirulina extract that has a concentration smaller than 10 μg/ml and is effective in treating TNF-α cytokine storm.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout.

In the accompanying drawings:

FIG. 1 is a high-level schematic illustration of cytokine storm (CS) and its treatment by the disclosed spirulina extracts, according to some embodiments of the invention.

FIG. 2 provides a comparative metabolomics profile indicating downregulated and upregulated compounds in disclosed spirulina extracts, according to some embodiments of the invention, compared with “solar” spirulina extracts grown under solar illumination.

FIGS. 3A-3C provide data that indicates the anti-inflammatory activity of the spirulina extracts that was measured with respect to TNF-α and IL-6 secreted from murine macrophage cell lines (RAW 264.7).

FIGS. 4A and 4B provide data that indicates the anti-inflammatory activity of the spirulina extracts that was measured with respect to TNF-α secreted from human monocyte cell lines (THP-1).

FIGS. 5A, 6A and 6B are high-level schematic illustrations of cultivation systems, according to some embodiments of the invention.

FIG. 5B is a high-level schematic flowchart illustrating cultivation, extraction and treatment methods, according to some embodiments of the invention.

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention may be practiced without the specific details presented herein. Furthermore, well known features may have been omitted or simplified in order not to obscure the present invention. With specific reference to the drawings, it is stressed that the particulars shown are by way of example and for purposes of illustrative discussion of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that may be practiced or carried out in various ways as well as to combinations of the disclosed embodiments. Also, it is to be understood that the phraseology and terminology employed herein are for the purpose of description and should not be regarded as limiting.

Spirulina extracts, sublingual spray formulation thereof, spraying devices for using the formulation, as well as methods of preparing and using the spirulina extracts to treat TNF-α related inflammation are provided. The spirulina extracts are prepared by water extraction of Arthrospira spp. biomass that is cultivated under controlled, ultra-high-density conditions with strong UV illumination and strong continuous mixing, and are characterized by high levels of c-phycocyanin, sorbitol and adenosine derivates—which were found to have a strong low-dose effect of reducing TNF-α secretion. The spirulina extracts may correspondingly be used to prevent or alleviate cytokine storm related to various infections or autoimmune diseases.

Disclosed spirulina extracts and/or fractionated compounds thereof, e.g., in form of sublingual spray formulations may enable treating a range of inflammatory conditions, including cytokine storm (CS), or hypercytokinemia, triggered by the macrophage activation syndrome (MAS) resulting from the novel coronavirus (SARS-CoV-2) or induced by other viral infections. Regarding CS, disclosed spirulina extracts, e.g., in form of sublingual spray formulations may prevent or alleviate CS as the main factor responsible for critical COVID-19 incidences, including acute respiratory distress syndrome (ARDS). Accordingly, disclosed spirulina extracts, e.g., in form of sublingual spray formulations may provide a treatment method that is agnostic to mutations in SARS-CoV-2 or other viral infections, and may be used as a complementary measure to the administration of vaccines, preventing severe COVID-19 illness and reducing admissions to intensive care units (ICU).

Disclosed spirulina extracts may further be used to treat other inflammatory conditions, especially conditions related to high macrophage- and monocyte-induced tumor necrosis factor (TNF)-α levels such as those associated with, e.g., heart failure, inflammatory bowel diseases (IBD) such as Crohn's disease, thrombophilia, gingivitis as well as CS related to autoimmune-related inflammatory diseases. Specifically, disclosed spirulina extracts, e.g., in form of sublingual spray formulations may decrease macrophage- and monocyte-induced TNF-α levels in patients suffering therefrom.

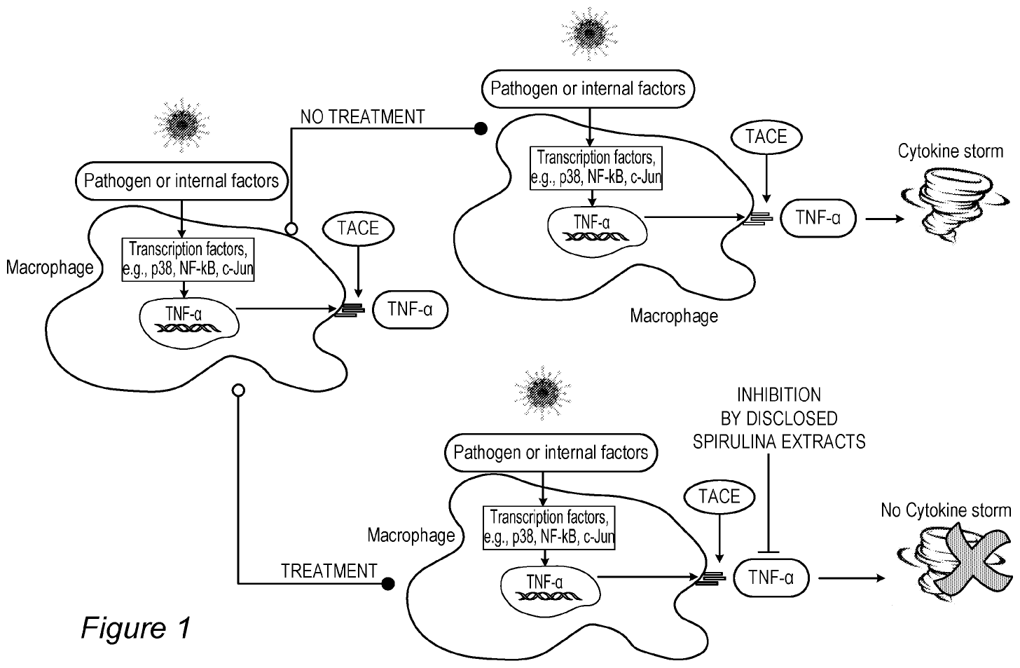

FIG. 1 is a high-level schematic illustration of cytokine storm (CS) and its treatment by the disclosed spirulina extracts, according to some embodiments of the invention. Pathogens (e.g., viruses such as SARS-CoV-2 or influenza virus, or various bacteria) or internal factors (e.g., autoimmune response) may interact with monocytes and/or macrophages to induce over-production of TNF-α and create a health threatening CS. Presence of disclosed spirulina extracts is shown herein to prevent or alleviate CS. For example, starting from the known cascade of cellular signals in macrophages, application of disclosed spirulina extracts may inhibit TNF-α by the macrophages, to prevent CS or alleviate its symptoms.

Two outstanding characteristics of disclosed spirulina extracts, which are prepared from Arthrospira spp. (e.g., A. platensis) that are cultivated under the disclosed photosynthetically controlled conditions, are the specific anti-TNF effects and the limited and capped range of effective concentrations (up to 10 μg/ml). These characteristics are unique and not present in other spirulina extracts or in cyanobacteria that are cultivated by other methods (such as low-density cultures with homogenous illumination and/or cultivation using solar illumination). Among other features, disclosed water-based extracts have upregulated bio-active compounds comprising c-phycocyanin, sorbitol and adenosine derivates, which may be the contributing factors to using the disclosed extracts as anti-inflammatory agents.

The following results illustrate these outstanding characteristics specifically, using LPS (lipopolysaccharides)-activated macrophages and monocytes, and comparing spirulina extracts from Arthrospira spp. cultivated under the disclosed photosynthetically controlled conditions with spirulina extracts from Arthrospira spp. cultivated under natural light conditions. Indeed, disclosed water-based extracts of photosynthetically controlled Arthrospira spp., at a concentration of 0.1 μg/ml, were found to decrease macrophage- and monocyte-induced TNF-α levels by over 70% and 40%, respectively, with concentration levels above 10 μg/ml of the spirulina extract lacking this activity. Accrodingly, it is suggested that treatment with disclosed spirulina extracts may lead to considerable reduction of COVID-CS and ARDS, and generally providing an effective anti-TNF therapy.

Non-limiting examples for the photosynthetically controlled conditions under which the Arthrospira spp. was cultivated include temperatures under 31±2° C., pH of 10.8±0.2 and irradiance of between 700-1,500 μmol/m²s, subranges thereof, or possibly even higher irradiance. Specifically, as disclosed below, cultivation of the Arthrospira spp. in an ultra-high-density culture having a density between 3 g/l and 10 g/l and under ultraviolet radiation intensity of between 70-150 μmol/m²s was found to yield the disclosed spirulina extracts. The water-based spirulina extracts may be produced by water-extraction and cycles of freezing and thawing applied to the cultivated Arthrospira spp.

The following experimental results were achieved as described in Tzachor et al. 2021, incorporated herein in its entirety—comparing disclosed extracts with “solar extracts” which represent spirulina extract for which the cyanobacteria were grown under solar illumination, according to the common practice. Growth conditions for disclosed extracts and solar extracts were similar, including the intensity of illumination (750 μmol/m²s), except for the spectral distribution of the illumination, including full-range solar spectrum for the solar extracts and red, blue and UV LED illumination for the disclosed extracts. The cyanobacteria (UTEX 3086) were cultivated under 31±2° C. and pH of 10.8±0.2 and both types of extracts were water-extracted using physical freeze-thawing for cellular disruption.

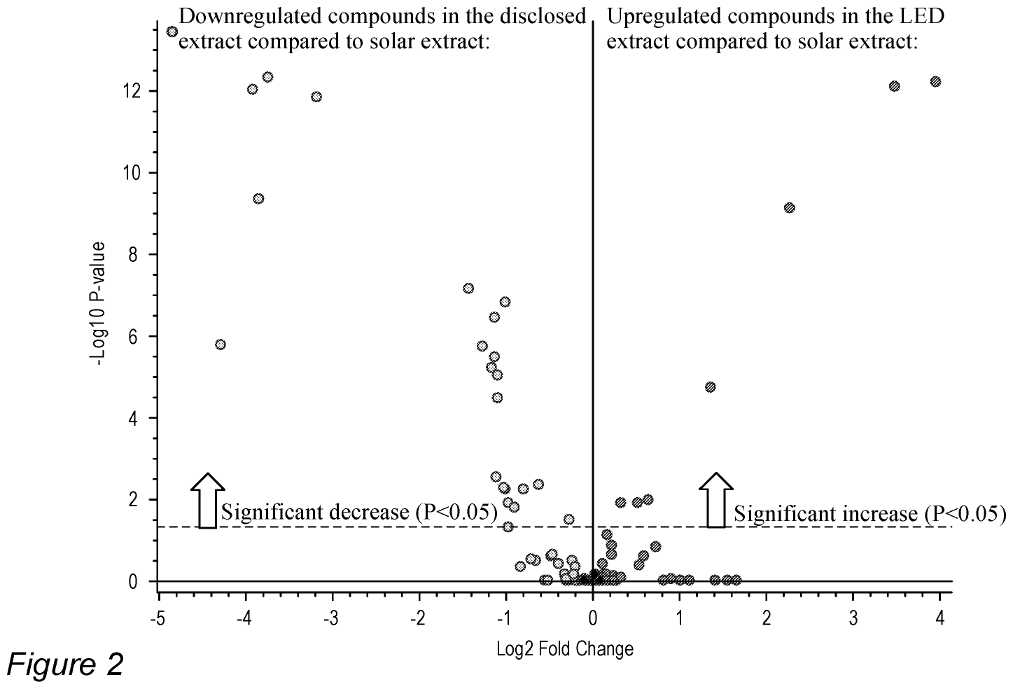

FIG. 2 provides a comparative metabolomics profile indicating downregulated and upregulated compounds in disclosed spirulina extracts, according to some embodiments of the invention, compared with “solar” spirulina extracts grown under solar illumination. The extracts were prepared, and the metabolomics profiles derived as described in Tzachor et al. 2021. The compounds were determined using liquid chromatography with tandem mass spectrometry (LC-MS-MS). The compounds are indicated as light-gray dots for downregulated compounds on the left-hand side of the diagram, and as dark-gray dots for upregulated compounds on the right-hand side of the diagram. Black dots indicate compounds which were expressed similarly in either extract. Compounds above the broken line presented statistically significant differences (P value <0.05 in a T-test) between the two types of extracts. Seven compounds were found to be significantly upregulated in the disclosed spirulina extract compared to solar spirulina extract while 23 compounds were found to be significantly downregulated. Two of the upregulated compounds were sorbitol and an adenosine derivate, having respectively 97% and 91% MS/MS spectral similarity compared to known databases. These bioactive compounds (having known anti-inflammatory properties) were significantly increased in the disclosed spirulina extract by factors of 1.7 and 4.8, respectively (with P values of 0.01 and 7.8·10⁻¹⁰, respectively). In addition, C-Phycocyanin (CPC) bioactive compound was also found to be significantly increased in the disclosed spirulina extract, by a factor of 4.7. (CPC levels were measured using standard spectrophotometric methods).

It is noted that while phycocyanin is found in both types of extracts and is known to somewhat reduce TNF-α secretion, the known effects are much smaller and at much high doses than those disclosed herein—indicated a synergistic effect of multiple upregulated compounds in inhibiting TNF-α secretion and CS that is outstanding in the presented results. Clearly, in order to maintain this composite synergistic effect, disclosed spirulina extracts must be produced from biomass cultivated under consistent and strictly controlled conditions (e.g., light composition, irradiation level, temperature, pH)., and irrespective of external conditions, such as disclosed herein.

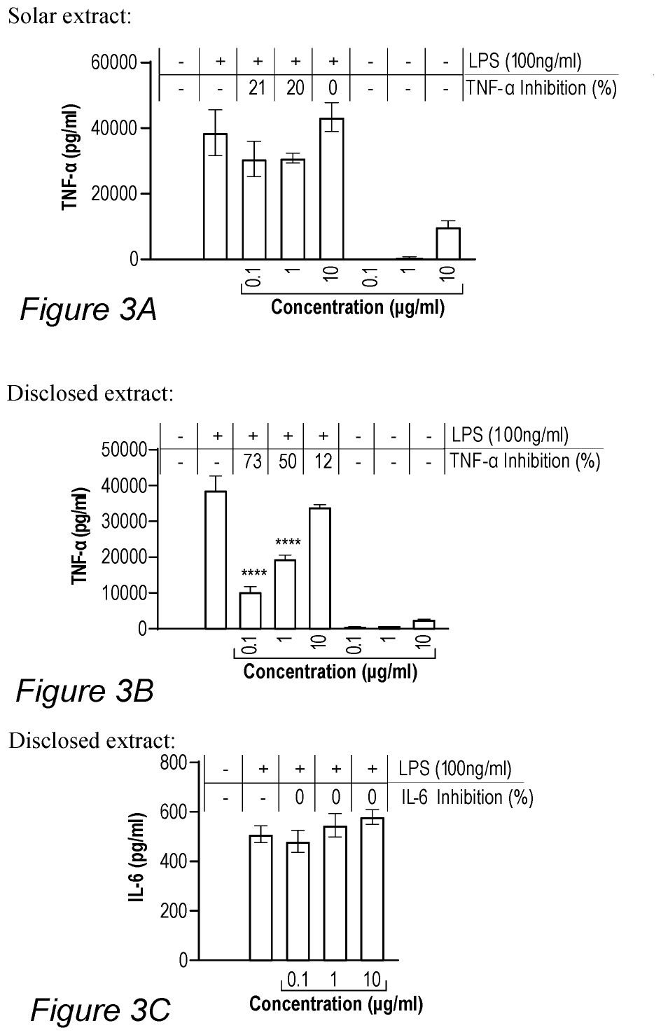

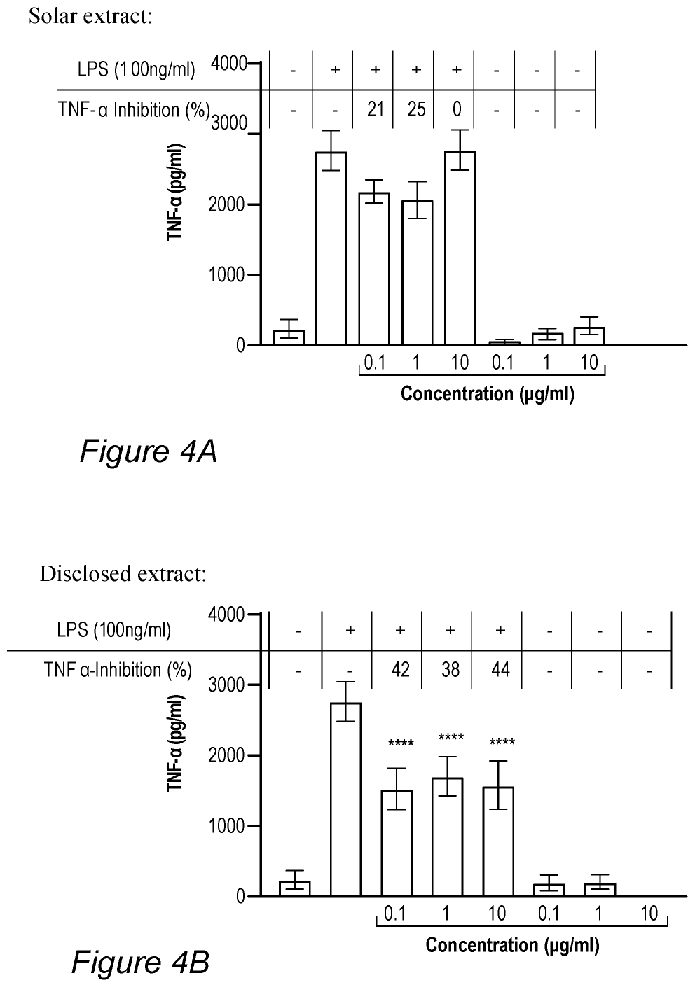

FIGS. 3A-3C provide data that indicates the anti-inflammatory activity of the spirulina extracts that was measured with respect to TNF-α and IL-6 secreted from murine macrophage cell lines (RAW 264.7). FIG. 3A provides results for solar spirulina extracts and FIGS. 3B, 3C provide results for disclosed spirulina extracts. FIGS. 4A and 4B provide data that indicates the anti-inflammatory activity of the spirulina extracts that was measured with respect to TNF-α secreted from human monocyte cell lines (THP-1). FIG. 4A provides results for solar spirulina extracts and FIG. 4B provides results for disclosed spirulina extracts.

In both cases the data measured using ELISA (enzyme-linked immunosorbent assay) kits, as described in Tzachor et al. 2021. Activation of the respective cell lines by LPS is indicated by “+” signs at the upper row for each graph (“−” data representing controls with non-activated cell lines) and TNF-α and IL-6 are indicated by the respective percentage of inhibition (“−” signs represent lack of inhibition for non-activated cell lines and for one activated control in each graph). The bars indicate means±SD (standard deviation) and **** indicates statistical significance with p<0.001 compared with the untreated LPS-activated cell lines.

Comparing FIG. 3B to FIG. 3A, disclosed spirulina extract exhibits a large and significant reduction in TNF-α secretion, reaching a 70% reduction for 0.1 μg/ml extract concentration and a 50% reduction for 1 μg/ml extract concentration (see FIG. 3B). Prior art spirulina extracts (here as solar extracts) do not yield such a large and significant reduction in TNF-α secretion (see FIG. 3A). Surprisingly, higher concentrations of disclosed spirulina extract of 10 μg/ml also do not yield such a large and significant reduction in TNF-α secretion (see FIG. 3B)—indicating an optimal effective concentration below 10 μg/ml, e.g., at 0.1 μg/ml.

It is noted that the anti-TNF-α effect exhibited by the disclosed spirulina extracts is not linearly dose-dependent but rather corresponds to a non-monotonic dose response curve (NMDRC) and is achieved by very low doses, suggesting effects on cellular endpoints such as cell proliferation and organ development through interaction with receptors.

The inventors note that these very low and specific extract concentrations enable administration of disclosed spirulina extracts sublingually, e.g., in a sublingual spray formulation comprising the disclosed spirulina extract and/or fractionated compounds thereof (and optionally a pharmaceutical acceptable carrier) to yield a concentration in the blood that is smaller than 10 μg/ml, e.g., of 0.1 μg/ml, or 1 μg/ml or intermediate values. For example, a corresponding spraying device may be configured to administer an amount of spray that yields a blood concentration of 0.1-1 μg/ml in the patient, e.g., in one or in a specified number of spraying actions. For example, an oral spray bottle that emits 0.14 ml per dose of spray, containing e.g., liquid with 0.4-4.0% (4.0-40 g/l) of the disclosed spirulina extract, could be used twice a day (every 12 hours) to maintain blood concentration (in adults) within the active range of 0.1-1 μg/ml throughout the day. In various embodiments, the spraying device may be configured to administer a dose of sprayed liquid of between 0.05 ml and 3 ml (or intermediate ranges, e.g., 0.05 ml-0.5 ml, 0.1 ml-1 ml, etc.), with correspondingly adjusted concentration of the active ingredients (spirulina extracts and/or compounds fractionated therefrom).

Moreover, concerning FIG. 3C, it is noted that disclosed spirulina extracts are specific to TNF-α secretion and do not affect secretion of IL-6, suggesting that disclosed spirulina extracts may provide a TNF-α specific inhibitor rather than a general inhibitor of the inflammatory process, which makes them particularly desirable for use as a specific anti-CS agent. It is noted that the disclosed extracts did not exhibit off target effects in the absence of LPS stimulation, suggesting their safety.

Comparing FIG. 4B to FIG. 4A provide data that indicates the anti-inflammatory activity of the spirulina extracts that was measured with respect to TNF-α secreted from THP-1 human monocyte cells. Disclosed spirulina extract exhibits a large and significant reduction in TNF-α secretion, of about a 40% reduction for the whole range of 0.1 μg/ml to 10 μg/ml extract concentration (see FIG. 4B). Prior art spirulina extracts (here as solar extracts) do not yield such a large and significant reduction in TNF-α secretion (see FIG. 4A). In contrast to the effects of macrophages, this effect on monocytes is constant over the range of concentrations.

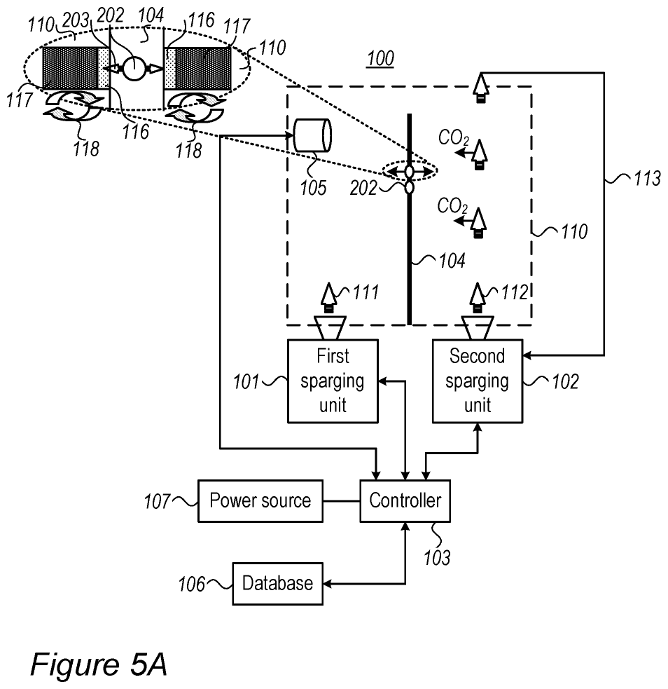

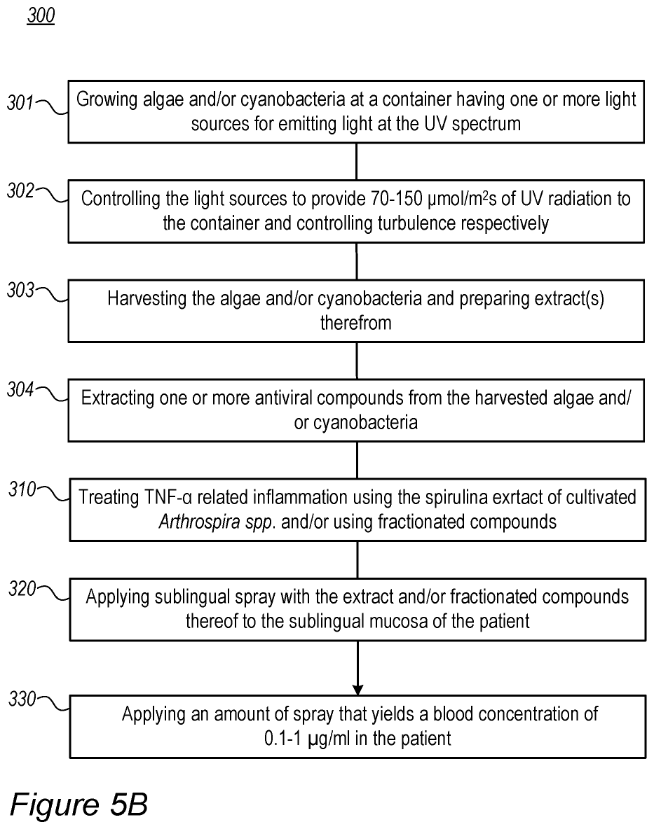

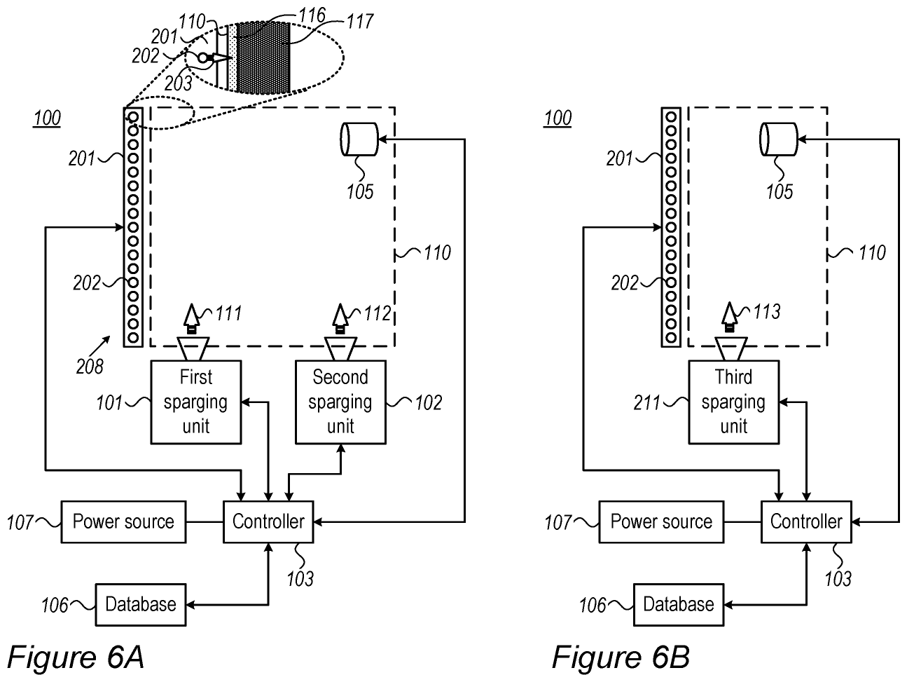

FIGS. 5A, 6A and 6B are high-level schematic illustrations of cultivation systems 100, according to some embodiments of the invention. FIG. 5B is a high-level schematic flowchart illustrating cultivation, extraction and treatment methods 300, according to some embodiments of the invention. Cultivation system 100 is configured to grow algae and/or cyanobacteria at high density and under high illumination intensity. Elements from FIGS. 5A, 6A and 6B may be combined in any operable combination, and the illustration of certain elements in certain figures and not in others merely serves an explanatory purpose and is non-limiting. It is noted that any disclosed value may be modified by ±10% of the value. The method stages may be carried out with respect to cultivation system 100 described herein, which may optionally be configured to implement method 300. Method 300 may be at least partially implemented by at least one computer processor, e.g., in controller 103 comprising corresponding processing unit(s). Certain embodiments comprise computer program products comprising a computer readable storage medium having computer readable program embodied therewith and configured to carry out the relevant stages of method 300. Method 300 may comprise the disclosed stages, irrespective of their order.

Cultivation system 100 comprises at least one first sparging unit 101 having a plurality of nozzles and configured to distribute a first predetermined fluid 111 (e.g., air and/or nitrogen bubbles) into a water-filled algae cultivation container 110 (e.g., a bio-reactor) at a first operating flow rate so as to allow mixing therein (indicated schematically by arrows 118). Cultivation system 100 may further comprise at least one second sparging unit 102 having a plurality of nozzles and configured to distribute a second predetermined fluid 112 (e.g., gas bubbles with CO₂, indicated schematically, and/or dissolved phosphorus for mass transfer) into container 110 at a second operating flow rate. Fluids exiting container 110, such as gas from second predetermined fluid 112 may be recycled 113 to fully utilize remaining CO₂ therein (illustrated schematically).

Cultivation system 100 may further comprise at least one controller 103 in communication with first and second sparging unit 101, 102, and configured to control the first operating flow rate and the second operating flow rate provided thereby. Controller 103 may comprise one or more processing units that implement computer code. For example, at least one nozzle of first sparging unit 101 and/or at least one nozzle of second sparging unit 102 may be configured to distribute fluid into cultivation container 110 based on instructions from at least one controller 103. In some embodiments, the first operating flow rate may be based on the second operating flow rate and/or at least one of the operating flow rates may be predetermined. In some embodiments, the first operating flow rate may be adapted to allow turbulent mixing of the algae in cultivation container 110. In some embodiments, the second operating flow rate may be adapted to allow mass transfer and/or assimilation of materials in a liquid in cultivation container 110. Information may flow between controller 103 and first and second sparging unit 101, 102, as well as between controller 103 and other elements in the system, as indicated schematically by the arrows.

Second predetermined fluid 112 may include gas bubbles with over 30% CO₂ concentration. The source for the first predetermined fluid(s) and/or for the second predetermined fluid(s) may be external to cultivation system 100, for example geothermal power stations may provide a source of dissolved carbon and/or sulfur for the second predetermined fluid.

The first operating flow rate of at least one nozzle of first sparging unit 101 (e.g., 100 ml/min) may be different from the second operating flow rate of at least one nozzle of second sparging unit 102 (e.g., 5 ml/min) In some embodiments, at least one nozzle of first sparging unit 101 may have a diameter larger than about 1 millimeter. In some embodiments, at least one nozzle of second sparging unit 102 may have a diameter smaller than about 1 millimeter. In some embodiments, nozzles of first sparging unit 101 as well as of second sparging unit 102 may distribute the same fluid (e.g., air), with nozzles of each sparging unit having different diameters.

Larger apertures of first sparging unit 101 may be configured to provide first predetermined fluid 111 with large bubbles (e.g., of air and/or nitrogen) to agitate and mix 118 the suspended biomass in container 110, while smaller apertures of second sparging unit 102 may be configured to provide second predetermined fluid 112 with small bubbles (e.g., of or comprising CO₂) to transfer CO₂ from the gas to the liquid to be accessible to the suspended biomass in container 110. Advantageously, the difference in the size of the delivered bubbles may prevent combination of the bubbles of streams 111, 112, providing simultaneously mixing 118 by the high throughput of big and fast bubbles in stream 111 and effective CO₂ supply by the small throughput of small and slow bubbles in stream 111.

Cultivation system 100 may further include a physical barrier 104 configured to separate the first fluid distributed by first sparging unit 101 and the second fluid distributed by second sparging unit 102 within cultivation container 110. In some embodiments, at least one nozzle of first sparging unit 101 and/or of second sparging unit 102 may be embedded into physical barrier 104 (not shown). In some embodiments, physical barrier 104 may be adapted to allow flow from one side of barrier 104 (having a first fluid distribution) to the other side of barrier 104 (having a second fluid distribution) at predefined (e.g., upper and lower) locations of cultivation container 110, in order to create a controlled flow within the container 110.

Cultivation container 110 with physical barrier 104 may include at least one light source 202 embedded into the physical barrier 104 such that container 110 may be illuminated (illumination denoted schematically by arrows 203) from within by from at least one light source 202 embedded into the physical barrier 104. Cultivation container 110 may include a plurality of physical barriers 104, each including at least one light source 202, such that a modular system may be created with algae and/or cyanobacteria growing between adjacent physical barriers 104, wherein at least one controller 103 may control illumination for all light sources 202 embedded into the physical barriers 104. In certain embodiments, cultivation system 100 may be configured to reach a very high density of the cultivated biomass with a corresponding small optical depth that yield a relatively thin illuminated zone 116 and a much thicker dark zone 117 in container 110, with the biomass being continuously agitated 118 (e.g., by strong bubbling of fluids 111 and/or 112) so that individual cells of algae and/or cyanobacteria have but a brief residence time in illuminated zone 116 before returning to dark zone 117. In non-limiting examples, the thickness of illuminated zone 116 may be configured to be between 0.1 cm and 1.5 cm, depending on the density of the suspension and the illumination density, and may be controlled by controller 103 and adjusted according to specified requirements. Accordingly, illumination 203 (and particularly UV components thereof) may be set at very high levels as the brief residence time prevents illumination damage to the individual cells.

Cultivation system 100 may further include at least one sensor 105 (e.g., a temperature sensor) coupled to controller 103 and configured to detect at least one feature within cultivation container 110. For example, at least one sensor 105 may be configured to detect any of the pH levels, the temperature, and the pressure conditions within cultivation container 110 and/or sections thereof. In some embodiments, at least one sensor 105 may also be configured to detect parameters external to cultivation container 110, for example measuring mass flow of the gas emissions from cultivation container 110 to determine an amount of substance that was absorbed in the algae cells by subtracting the emitted amount from the amount inserted into the container (e.g., by second sparging unit 102).

Cultivation system 100 may further include at least one database 106 (and/or memory unit) configured to store algorithms for operation of controller 103, for instance database of operating rates for each nozzle and/or each sparging unit. In some embodiments, cultivation system 100 may further include a power source 107 coupled to controller 103 and configured to provide electrical power to cultivation system 100. Power source 107 may be configured to power at least one first sparging unit 101 and at least one second sparging unit 102, e.g., to operate at different rates.

Data gathered by at least one sensor 105 may be analyzed by controller (or processor) 103 to detects if an attribute exceeds a predetermined threshold, for instance threshold for pH level and/or temperature and/or CO₂ concentration within the container 110. In case that conditions within cultivation container 110 (e.g., as detected by sensor 105) exceed at least one threshold, then controller 103 may operate at least one nozzle of first sparging unit 101 and/or at least one nozzle of second sparging unit 102 at a different flow rate. For example, detecting CO₂ concentration within the container 110 exceeds 40% (or detecting low pH levels) may cause at least one nozzle of second sparging unit 102 to lower flow rate of second sparging unit 102 to ˜2 millimeters/minute. In some embodiments, at least one nozzle of second sparging unit 102 may operate only upon receiving a signal from sensor 105 that an attribute exceeds a predetermined threshold, and not operated in a constant rate.

At least one nozzle of first sparging unit 101 may be configured to operate only upon receiving a signal from sensor 105 that an attribute exceeds a predetermined threshold, for example increasing mixing flow 118 as the density of algal population increases. At least one nozzle of first sparging unit 101 and/or at least one nozzle of second sparging unit 102 may operate in a constant rate, continuously, or possibly intermittently. At least one nozzle of first sparging unit 101 and/or at least one nozzle of second sparging unit 102 may operate in a non-constant rate continuously, or possibly intermittently.

Cultivation container 110 may comprise a bubble column configuration with at least one first sparging unit 101 and at least one second sparging unit 102 positioned on the same surface of the bubble column container. Cultivation container 110 may have an airlift configuration with at least one second sparging unit 102 positioned at a bottom portion of a down-comer that may be distal to sensor 105, such that residence time of bubbles from at least one second sparging unit 102 may be increased.

Cultivation system 100 may be configured to enable maintaining at least 20% organic carbon within container 110, calculated in addition to carbon provided as CO₂ bubbles. In some embodiments, at least portion(s) of the algae within container 110 may comprise any photosynthetic microorganism such as algae and/or cyanobacteria used to prepare spirulina preparations, including, e.g., Arthrospira platensis, A. fusiformis and/or A. maxima.

As illustrated schematically in FIG. 5B, a method 300 of growing algae and/or cyanobacteria in cultivation system 100 may comprise growing algae and/or cyanobacteria in a container having one or more light sources for emitting light at the UV spectrum (stage 301). Illumination may be provided by at least one of light sources 202, configured to emit UV light at both UVA and UVB spectra. In some embodiments, the ratio between the emitted intensities of UVA/UVB radiation may be in a range of 10-15, for example, 10 UBA/UVB. In certain embodiments, method 300 may be used to grow cyanobacteria from which spirulina extracts are produced, e.g., Arthrospira spp. Method 300 may further comprise providing the UV radiation at intensities of 1,000-10,000 kJ/m² (stage 302), for example, 5000 kJ/m² or at any other intermediate value. For example, controller 103 may be configured to control the provision of the UV radiation using on/off radiation pulses. In some embodiments, each pulse may last 0.0099 sec and between 1-100 of such pulses may be provided per second, for example, 10 times per second. It is noted that ca. 0.01 sec of 1,000 kJ/m² illumination yields about ten times the intensity of solar UV radiation, which changes the chemical composition of the algae and/or cyanobacteria to yield extract compositions disclosed below.

In some embodiments, optimized controlled provision of the harmful UV radiation may allow increasing the amounts of antiviral compounds in Arthrospira spp. And the extracted spirulina while avoiding damaging the growing algae or the growing rate. In some embodiments, the on/off nature of the radiation provision may allow controlling the amount of harmful radiation provided. Moreover, the constant mixing and/or bubbling 118 of the suspension in container (by sparging units 101 and/or 102) ensures only brief exposures of any individual algal or cyanobacterial cells to the intense radiation, preventing photoinhibition and damage to the cells. Controller 103 may be respectively configured to control the extent of turbulence provided by sparging units 101 and/or 102 to avoid radiation damage to the cells (stage 302). For example, method 300 and cultivation system 100 may be configured to achieve the required UV photo-modulation in a thin-film cultivation system, by turning the UV light source on and off and/or by creating shade patterns that yield intermittent illumination of the algae and/or cyanobacteria. In bubbled cultivation systems 100, the relative velocities of the flows of the culture suspension and of the gas bubbles relative to the UV light source may be controlled to achieve specified patterns of on/off UV exposure cycles.

Method 300 further comprises harvesting the algae and/or cyanobacteria (stage 303), e.g., implementing continuous harvesting and matching the harvest rate to the growth rate. Method 300 further comprises preparing extract(s) from the harvested algae and/or cyanobacteria (stage 303), e.g., by applying one or more freeze-thaw cycles to break the cell walls and enhance the extractability of the suspension. For example, the harvested biomass may be rapidly frozen to −20° C. then thawed at 0 to 4° C. until completely de-frosted.

Method 300 may further comprise extracting at least one antiviral compound from the biomass (stage 304). For example, antiviral compounds were shown to be extractable from biomass of Arthrospira spp., cultivated and harvested as disclosed herein, to yield spirulina anti-viral extracts and/or preparations. For example, a wet biomass of spirulina may be suspended in pure water (hot or cold) to obtain a product with 10 weight % dry substance. The insoluble substances may be removed by continuous centrifugation. The supernatant, containing soluble biologically active substances may be used as an antiviral extract. Spirulina antiviral extract comprises water-soluble pigments (e.g., phycocyanin), proteins. nucleic acids, polysaccharides and ash. These compounds may be further fractionated (e.g., by chromatography and ethanol precipitation) in order to enhance antiviral activity. While in some embodiments the harvested biomass may be used directly, e.g., orally, advantageously using the extract and/or fractionated compounds thereof allows using smaller amounts for daily consumption and enables the usage of sublingual/oral sprays rather that consumption via the digestive track that might affect the efficacy of the active compounds.

The inventors have found out that disclosed spirulina extracts have enhanced antiviral activity against pathogenic viruses such as HSV-1, HSV-2, human cytomegalovirus, influenza and COVID-19 virus, with respect to spirulina extracts from cyanobacteria cultivated under prior art conditions which typically include solar illumination at lower intensity and lower density of the cyanobacterial in the bioreactor. Oral administration of the spirulina extract (e.g., 1-3 g dry weight per person per day) was found to prevent viral infestation as well as to relieve symptoms of viral diseases and shortening recovering time.

Accordingly, and in view of the results disclosed above, method 300 may further comprise treating TNF-α related inflammation (stage 310) by applying sublingual spray formulation comprising the disclosed spirulina extract and/or fractionated compounds having a concentration smaller than 10 μg/ml (e.g., 0.1 μg/ml, 1 μg/ml or intermediate values) to the sublingual mucosa of a patient suffering from inflammation (stage 320), e.g., by applying an amount of spray that yields a blood concentration of 0.1-1 μg/ml in the patient (stage 330). Treating TNF-α related inflammation 310 may be carried out by using the spirulina extract and/or using fractionated compounds as an anti-inflammatory agent to decrease macrophage- and monocyte-induced tumor necrosis factor TNF-α levels, e.g., to treat acute respiratory distress syndrome (ARDS), heart failure, Crohn disease, thrombophilia and/or gingivitis.

FIGS. 6A and 6B illustrates schematically embodiments of cultivation systems 100, according to some embodiments of the invention. Cultivation system 100 may comprise at least one illumination unit 201 coupled to controller 103, to illuminate cultivation container 110. Illumination unit(s) 201 and controller 103 (or another controller) may be included in a bioreactor illumination system 208 for growing algae and/or cyanobacteria. The distance between cultivation container 110 and illumination unit(s) 201 may be modified to control the illumination received by cultivation container 110. For example, bringing illumination unit(s) 201 closer to cultivation container 110 to increase illumination of the culture therein. The distance between cultivation container 110 and illumination unit(s) 201 may be controlled by controller 103, for example, included in illumination system 208. In addition to, or instead of, changing the distance of illumination unit 201 from cultivation container 110, the illumination intensity of light sources 202 in illumination unit 201 may be controlled. Illumination unit 201 may include at least one light source 202 (e.g., LED) such that each light source 202 may be controlled separately by controller 103. In some embodiments, one or more light source(s) 202 may be controlled to illuminate with a different intensity than another light source(s) 202. All light sources 202 may be controlled to change the illumination intensity, either manually or according to preset timing and/or sensed conditions in cultivation container 110. At least some of light sources 202 may be configured to emit light in the UV spectrum, for example, in both the UVA and the UVB range. The ratio between the emitted radiation in UVA and UVB (UVA/UVB ratio) may be between 10 and 15, e.g., 10, 12, 14, 15 UBA/UVB or have intermediate values. The UV radiation may be provided at 1,000-10,000 kJ/m², for example, 2000 kJ/m², 5000 kJ/m², 7000 kJ/m², 9000 kJ/m² or any other intermediate value. Controller 103 may be configured to control the provision of the UV radiation using radiation pulses, e.g., each pulse may last less than 0.01 sec, e.g., 0.008, 0.009, 0.0095 or 0.0099 seconds, or any intermediate value, and between 1-100 of these pulses may be provided per second, for example, 10 times per second. It is noted that ca. 0.01 sec of 1,000 kJ/m² illumination yields about ten times the intensity of solar UV radiation, which changes the chemical composition of the algae and/or cyanobacteria to yield extract compositions disclosed below. Some light sources 202 may be configured to emit light at the visible spectrum (e.g., wavelength of 400-700 nm).

The amount of light delivered to cultivation container 110 may be defined as an average of light flux delivered to the surface of cultivation container 110. Cultivation system 100 may be used to support ultra-high-density cultures (e.g., having a biomass density of 1, 5 or up to 10 g/l, or intermediate values), with illumination unit(s) 201 configured to have a light distribution of light source(s) 202 that provides an average light flux per algae/cyanobacteria cell that is comparable to average light flux per cell of low-density cultures achieving a similar level of average illumination per cell. Light intensity within cultivation container 110 may be measured with at least one sensor 105 and adjusted by controller 103. For example, for ultra-high-density cultures a typical thickness of illuminated zone 116 may range, e.g., between 1 mm and 5 mm, while a typical thickness of dark zone 117 may range, e.g., between 20 mm and 30 mm) The distance of illumination unit 201 from the side of container 110 may be adjusted with respect to the prevailing optical thickness (or optical depth, OD) of the biomass suspension to avoid photoinhibition and/or photo-bleaching. For example, at initial cultivation stages, when the culture density is relatively low, illumination unit 201 may be initially kept at a distance from the side of container 110 to biomass growth, while at later cultivation stages, once the OD increases, illumination unit 201 may be brought closer to the side of container 110 to promote biomass growth.

Ultra-high-density cultures be continuously and/or intermittently mixed and/or agitated (indicated schematically by arrows 118 in FIG. 5A), by mechanical means and/or by strong bubbling of fluids 111 and/or 112, to cause the algae and/or cyanobacteria to move between illuminated zone 116 and dark zone 117 and prevent damage to the cells, yielding illumination cycles for the algae/cyanobacteria (between illuminated zone 116 and dark zone 117) due to the short light passage. Ultra-high-density cultures may be illuminated with various wavelengths since in such biomass densities the wavelength may have nearly no effect on the growth due to the short light passage. This is in contrast to the common practice, according to which algae are illumination with specific wavelengths (e.g., with blue light) for normal growth since algae should respond to light differently. However, the inventors have found out experimentally that illumination with any wavelength may be used for ultra-high-density cultures.

The light penetration into cultivation container 110 may correspond to at least one of the light intensity, the light wavelength, the specific algal strain, and/or the algal culture density. It should be noted that the light penetration into cultivation container 110 may determine the volumetric ratio between illuminated zones 116 and dark zones 117 within cultivation container 110, and thus may affect the light intensity provided by illumination units 201, the gas flow rate through first sparging unit 101, the gas flow rate through second sparging unit 102, etc.—which may be adjusted and optimized respectively.

In some embodiments, cultivation container 110 may be illuminated by illumination unit(s) 201 to provide a daily amount of over 90% of maximal algae growth within the cultivation container 110. In some embodiments, illumination unit(s) 201 may be configured to illuminate the suspension in container 110 in a non-homogenous manner, e.g., using few high intensity light sources 202 spaced apart, because the illumination of the cells is local and temporally controlled (through agitation 118). The inventors have found out that high intensity intermittent illumination actually enhances growth of algae and/or cyanobacteria, over common practice configurations with homogenous distribution of low intensity light sources.

For example, the illumination photon flux density of at least one light source 202 may be 1200 nmole/m²s. In various embodiments, the photon flux density may range between 1000-1500 nmole/m²s, or have intermediate values. In some embodiments, illumination unit(s) 201 may include at least four light sources 202 per m². As an illustrative non-limiting example, illumination unit 201 having a surface area of about 6 m², a light path (thickness of illuminated zone 116) of about 4 cm may include 24 LED light sources 202, each having light flux of 1200 nmole/m²s. In some embodiments, at least a portion of the cyanobacteria within container 110 comprises Arthrospira spp. used to prepare spirulina extracts.

Controller 103 may be configured to control the illumination wavelength of the at least one light source 202, for instance with a dedicated illumination module adapted to modify the wavelength of the emitted illumination. In some embodiments, a constant temperature of 27° C. may be maintained within the container 110. In some embodiments, controller 103 may be configured to control at least one light source 202 to illuminate with a wavelength of 650 nanometers. It should be noted that according to common practice algae are illuminated with a particular wavelength (e.g., with blue light) for optimal growth, however experiments conducted by the inventors have shown that illumination with other wavelengths (e.g., with red light) may be used for enhanced growth.

As illustrated schematically in FIG. 6B, cultivation system 100 may be configured to operate with a single sparging unit, denoted in FIG. 6B as third sparging unit 211. Cultivation system 100 may further comprise illumination unit(s) 201, third sparging unit 211 may be configured to distribute a predetermined fluid 113 into cultivation container 110. Predetermined fluid 113 may comprise one or both of predetermined fluids 111, 112, or various mixtures of parts thereof. For example, third sparging unit 211 may include at least one nozzle to distribute first predetermined fluid 111 and at least one nozzle (e.g., having a different diameter) to distribute second predetermined fluid 112, which in combination are denoted schematically in a non-limiting manner as predetermined fluid 113. Third sparging unit 211 may be configured to generate turbulent mixing of the algae and/or cyanobacteria in cultivation container 110, as well as provide CO₂ for assimilation thereby, e.g., as disclosed with reference to FIGS. 5A and 6A.

In the above description, an embodiment is an example or implementation of the invention. The various appearances of “one embodiment”, “an embodiment”, “certain embodiments” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment. Certain embodiments of the invention may include features from different embodiments disclosed above, and certain embodiments may incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A spirulina extract comprising a water-based extract of Arthrospira spp., cultivated under photosynthetically controlled conditions to yield upregulated bio-active compounds comprising c-phycocyanin, sorbitol and adenosine derivates, wherein the spirulina extract has a concentration smaller than 10 μg/ml and is effective as an anti-inflammatory agent.

2. The spirulina extract of claim 1, wherein the photosynthetically controlled conditions comprise cultivating the Arthrospira spp. under 31±2° C., at pH of 10.8±0.2 and under irradiance of between 700 and 1500 μmol/(m²s).

3. The spirulina extract of claim 1, wherein the photosynthetically controlled conditions comprise cultivating the Arthrospira spp. in a ultra-high-density culture having a density between 3 g/l and 10 g/l and under ultraviolet radiation intensity of between 70-150 μmol/m²s.

4. The spirulina extract of claim 1, wherein the water-based extract is produced by water-extraction and cycles of freezing and thawing applied to the cultivated Arthrospira spp.

5. A pharmaceutical composition comprising fractionated compounds of the spirulina extract of claim 1.

6. Use of the spirulina extract of claim 1, or of fractionated compounds thereof, to decrease macrophage- and monocyte-induced tumor necrosis factor (TNF)-α levels.

7. (canceled)

8. The use of claim 6, for treating at least one of: virus-induced TNF-α cytokine storm, acute respiratory distress syndrome (ARDS), heart failure, Crohn disease, thrombophilia and gingivitis.

9. A sublingual spray formulation comprising the spirulina extract of claim 1, or of fractionated compounds thereof, having a concentration smaller than 10 μg/ml, and optionally further comprising a pharmaceutical acceptable carrier.

10. (canceled)

11. The sublingual spray formulation of claim 9, having a concentration of 0.4-4.0% by volume of the spirulina extract.

12. A method of treating TNF-α related inflammation, the method comprising applying the sublingual spray formulation of claim 9, to the sublingual mucosa of a patient suffering from inflammation.

13. The method of claim 12, further comprising applying an amount of spray that yields a blood concentration of 0.1-1 μg/ml in the patient.

14. A spraying device configured to administer the sublingual spray formulation of claim 9 to the sublingual mucosa of a patient suffering from inflammation.

15. The spraying device of claim 14, further configured to enable administration of an amount of spray that yields a blood concentration of 0.1-1 μg/ml in the patient, in one or in a specified number of spraying actions.

16. The spraying device of claim 15, wherein the sublingual spray formulation has a concentration of 0.4-4.0% by volume of the spirulina extract, and the spraying device is further configured to deliver between 0.05 ml and 0.5 ml per spraying action.

17. A method of preparing a spirulina extract, the method comprising:
cultivating Arthrospira spp. cyanobacteria, cultivated under photosynthetically controlled conditions to yield upregulated bio-active compounds comprising c-phycocyanin, sorbitol and adenosine derivates,
preparing a water-based extract of the cultivated Arthrospira spp. cyanobacteria to yield a spirulina extract that has a concentration smaller than 10 μg/ml and is effective in treating TNF-α cytokine storm.

18. The method of claim 17, wherein the cultivation of the Arthrospira spp. is carried out under 31±2° C., at pH of 10.8±0.2 and under irradiance of between 700 and 1500 μmol/(m²s).

19. The method of claim 17, wherein the cultivation of the Arthrospira spp. is carried out in a ultra-high-density culture having a density between 3 g/l and 10 g/l and under ultraviolet radiation intensity of between 70-150 μmol/m²s.

20. The method of claim 17, wherein the water-based extract is prepared by water-extraction and cycles of freezing and thawing applied to the cultivated Arthrospira spp.

21. The method of claim 17, further comprising using the spirulina extract as an anti-inflammatory agent to decrease macrophage- and monocyte-induced TNF-α levels.

22. The method of claim 21, further comprising treating at least one of: acute respiratory distress syndrome (ARDS), heart failure, Crohn disease, thrombophilia and gingivitis.
